(19) United States
(12) Patent Application Publication
(10) Pub. No.: US 2014/0377169 A1
(43) Pub. Date: Dec. 25, 2014

(54) Automated synthesis device to produce Re-188-Liposome and method thereof

(71) Applicants: Ming-Hsin Li, Taoyuan County (TW); Shu-Pei Chiu, Taoyuan County (TW); Chih-Hsien Chang, Taoyuan County (TW); Te-Wei Lee, Taoyuan County (TW)

(72) Inventors: Ming-Hsin Li, Taoyuan County (TW); Shu-Pei Chiu, Taoyuan County (TW); Chih-Hsien Chang, Taoyuan County (TW); Te-Wei Lee, Taoyuan County (TW)

(21) Appl. No.: 13/921,217

(22) Filed: Jun. 19, 2013

(51) Int. Cl. B01J 19/00; A61K 51/12

(52) U.S. Cl. CPC B01J 19/004; A61K 51/1234
USPC 424/1.21; 422/159

(57) ABSTRACT

An automated synthesis device to produce Re-188-BMEDA solution including: a plurality of reagent vials, three-way solenoid valves, gel filtration columns and micro pumps, and a reaction vial, a product vial, a temporary storage vial, a filter membrane, and a waste vial, wherein the plurality of reagent vials include first reagent vial and second reagent vials being connected to the reaction vial through first micro pump, the third reagent vial and fourth reagent vial being connected to the reaction vial through second micro pump, fifth reagent vial being connected to the reaction vial through third micro pump, and sixth reagent vial being connected to the temporary storage vial through fourth micro pump, wherein the reaction vial is connected to the plurality of gel filtration columns through the micro-pump, respectively. The automated synthesis device is operable with program to upgrade yield and avoid contamination.

### BACKGROUND OF THE INVENTION

1. Field of the Invention

The present invention relates to an automated synthesis device and method to produce Re-188-Liposome (rhenium-188-liposome), and more particularly to synthesis of rhenium-188-liposome using automated synthesis devices to obtain liposome encapsulated rhenium-188 for the purpose of accumulation of medicine in parts of tumors to improve therapeutic effect of nuclear medicine.

2. Description of Related Art

The artificial radioisotope Re-188 with half-life of 16.9 hours has been proved its enhanced permeability and retention (Enhanced Permeability and Retention, EPR) effect for accumulating in specific tumor tissue, transferring radioactive NANO-medicine to the tumor cell angiogenesis, blocking the path of the supply of nutrients and releasing rhenium-188 beta rays to kill cancer cells to achieve the purpose of cancer treatment. Also, it is confirmed to be effective for treatments of tumor metastases in animal experiments, including colon cancer, lung metastases and ascites metastases of colorectal cancer in tumors transfer mode.

Rhenium-188-Liposome as a metastatic tumor medicine treatment and diagnosis draws a high degree of attention recently. Liposome has a structure of phospholipid bilayer having same composition as cell membranes, good biocompatibility, and capable of decomposition in vivo. Liposome has a particle size between tens of nanometers (nm) to tens of microns (um), when the liposome enters the vessel of human body, because of the small gap of vascular endothelial cell of normal blood vessel tissues, the liposome is not able to enter; but tumor angiogenesis in vascular endothelial cell has relative large gap and due to effect of EPR (Enhanced Permeability and Retention) the liposome is able to enter through gap into the tumor vascular cell, allowing mass liposome accumulation at the tumor site to enhance medications efficiency. Rhenium-188 nuclide can be embedded in the liposome by chelating agent BMEDA for developing radioactive NANO medicine a rhenium-188 lipsome (Re-188-Liposome). Rhenium-188 is a radioisotope capable of diagnosis and treatment with its suitable half-life (16.9 hours), and can radiate 155 thousand electron volts (keV) gamma rays for nuclear medicine diagnostic imaging applications, and also it can release beta energy up to 2.12 million electron volts (MeV) suitable for use as nuclear medicine treatment of cancer. This characteristic of Re-188 nuclides has triggered research in the aspect of application for imaging diagnosis and treatment of tumors.

Conventional synthesis method of Rhenium-188-Liposome medicine for diagnosis and treatment is achieved in manual. The convention method includes first conveying the rhenium-188 eluent directly into the reaction vial, heating the reaction solvent, importing into the pre-treated PD-10 gel filtration column to proceed with manual purification, and followed by pharmacokinetic studies and clinical assessment. Besides rhenium-188-Liposome labeling accomplished with manually operational method, other automatic labeling hardware includes process flow, structure, layout and arrangement are still having rooms for improvement to provide a unique and complete functionality. In the part of software, there is still remained high degree of manipulation complexity and bulk software in operation. Without proper improvement of above drawbacks may result in reduced yield and waste of resources.

### SUMMARY OF THE INVENTION

The main objective of the present invention is to provide an automated synthesis method for Rhenium-188-Liposome, including four stages in order, namely, labeling reaction, embedding reaction, separation and purification, and medication filtration. The automated synthesis method and its operational process for Rhenium-188-Liposome include hardware and software, but in the traditional automatic synthesis box for labeling, the software is usually attached to and controlled by other equipments. The automated synthesis device to produce Re-188-Liposome of the present invention aims to replace the traditional automatic synthesis box for labeling, which is attached to other equipments, with an one-touch or one-button press control. When the customized labeling process is in place, the process control software is burnt into a chip for compactness for the control process. The improvement of the control process can solve problems of complexity and bulk software adherent to the traditional manipulation process.

Another objective of the present invention is to provide an automated synthesis device, including the configuration of each unit module required to accomplish the reactions with the automated synthesis method of the present invention. The reactions include labeling, embedding, separation purification, and medication filtration in four stages. The reactions are processed with an automatic operation controlled by a computer software burn in-chip. To use the automated synthesis device of the present invention, it only needs to place the medications for reaction into specific reaction vial, turn on power to activate the operating system, and the preparation of the medication can be completed in a short time for clinic diagnosis and treatment.

### BRIEF DESCRIPTION OF THE DRAWINGS

FIG. 1 is a block diagram of the production of Re-188-BMEDA solution of the present invention;

FIG. 2 is schematic diagram showing a preferred embodiment of synthesis method of Re-188-BMEDA solution of the present invention, and

FIG. 3 is schematic diagram showing a preferred embodiment of synthesis device to produce Re-188-BMEDA solution of the present invention.

### DETAILED DESCRIPTION OF THE PREFERRED EMBODIMENT

The present invention relates to a metastatic tumor medicine treatment and diagnosis to provide an automated synthesis device and method to produce Rhenium-188-Liposome.

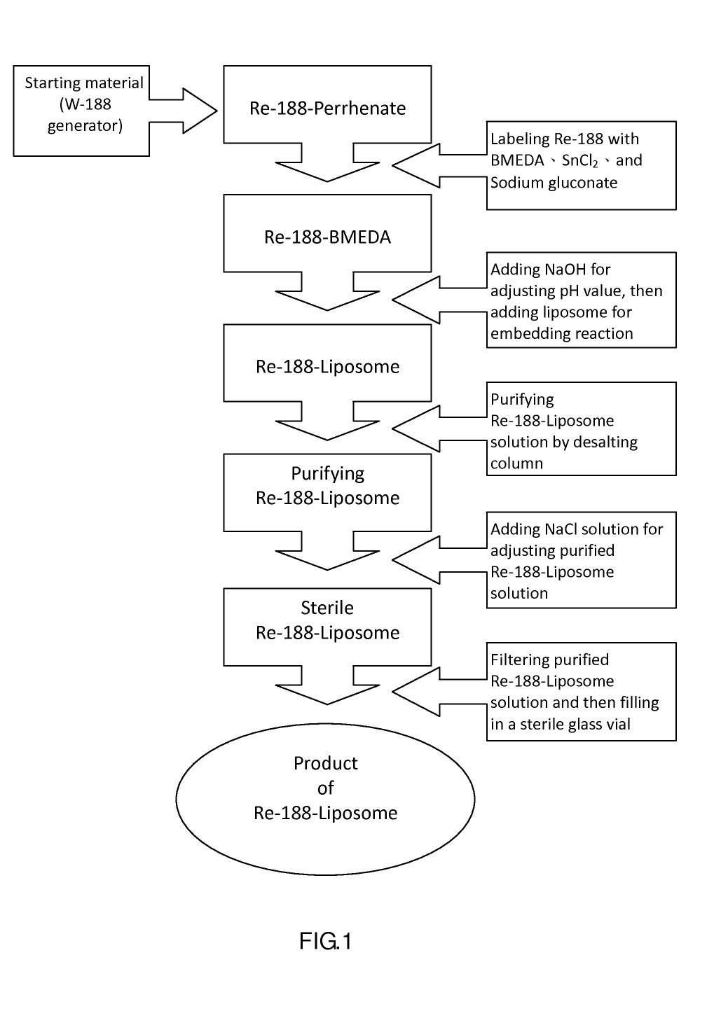

In FIG. 1, a block diagram showing the production of Re-188-BMEDA solution of the present invention includes four stages, comprising steps:

Stage 1: Labeling Reaction

Step 1a: Charging 0.68M sodium gluconate containing 10% glacial acetic acid solution, N,N-bis(2-mercapto ethyl)—N′,N′-diethylethylenediamine, referred to as BMEDA, and the solution of stannous chloride (SnCl2) with 10 mg/m modulation ratio into reaction vial G1, and then adding Re-188 solution with radioactivity of approximately 65 mCi˜470 mCi to the reaction vial G1 from the first reagent vial R1, the reaction taking place at temperature about 80° C. with a stirrer rotating at speed 100˜500 rpm to complete the reaction in the reaction vial G1, and the labeling reaction is accomplished with the solution of rhenium-188-BMEDA.

Stage 2: Embedding reaction

Step 2a: adding sodium hydroxide from the second reagent vial R2 to reaction vial G1, mixing with rhenium-188-BMEDA solution obtained in step 1a, and stirring the vial G1 with a stirrer rotating at speed 100-500 rpm to assure uniformity of mixing.

Step 2b: adding liposome from third reagent bottle R3 to the reaction vial in G1, heating the reaction vial G1 to about 60° C. and stirring the reaction vial G1 with a stirrer rotating at speed 100˜500 rpm.

Step 2c: After completion of the reaction, placing the reaction vial G1 at room temperature environment for cooling and the 188Re-Liposome solution is obtained.

Stage 3: Separation and purification

Step 3a: first using 0.9% sodium chloride injection for washing first to third PD-10 gel filtration column C1-C3, respectively.

Step 3b: adding an equal amount of rhenium-188-liposome solution from the reaction vial G1 to each of PD-10 gel filtration column C1-C3, respectively, and after the Re-188-liposome solution is completely discharged out of each of the PD-10 gel filtration column C1-C3, then adding an equal volume of 0.9% sodium chloride injection to each of PD-10 gel filtration column C1-C3 from the fourth reagent vial R4, respectively, and subject to the sodium chloride solution is fully discharged as waste solution, conveying the waste solution into waste vial W1 through the sixth solenoid valve V6.

Step 3c: after the waste solution being completely discharged, adding an equal volume of 2.5 mL of 0.9% sodium chloride injection to each of PD-10 gel filtration column C1-C3 from the fifth reagent vial R5, respectively.

Step 3d: conveying the discharged about 7.5 mL sodium chloride injection through sixth solenoid valve V6 into temporary storage vial T1 to complete the process of separation and purification.

Stage 4: medication and filtration

Step 4a: adding 6.5 mL of 0.9% sodium chloride injection from sixth reagent vial R6 through fourth pump P4 to the temporary storage vial T1 to mix with the solution obtained in Step 3d.

Step 4b: filtering the solution about 14 mL obtained from step 4a through micro pump P8, solenoid valve V7, and a sterile membrane F1 of thickness about 0.2 μm, and filling in a sterile glass product vial RL1 as a finished product of rhenium-188-Liposome.

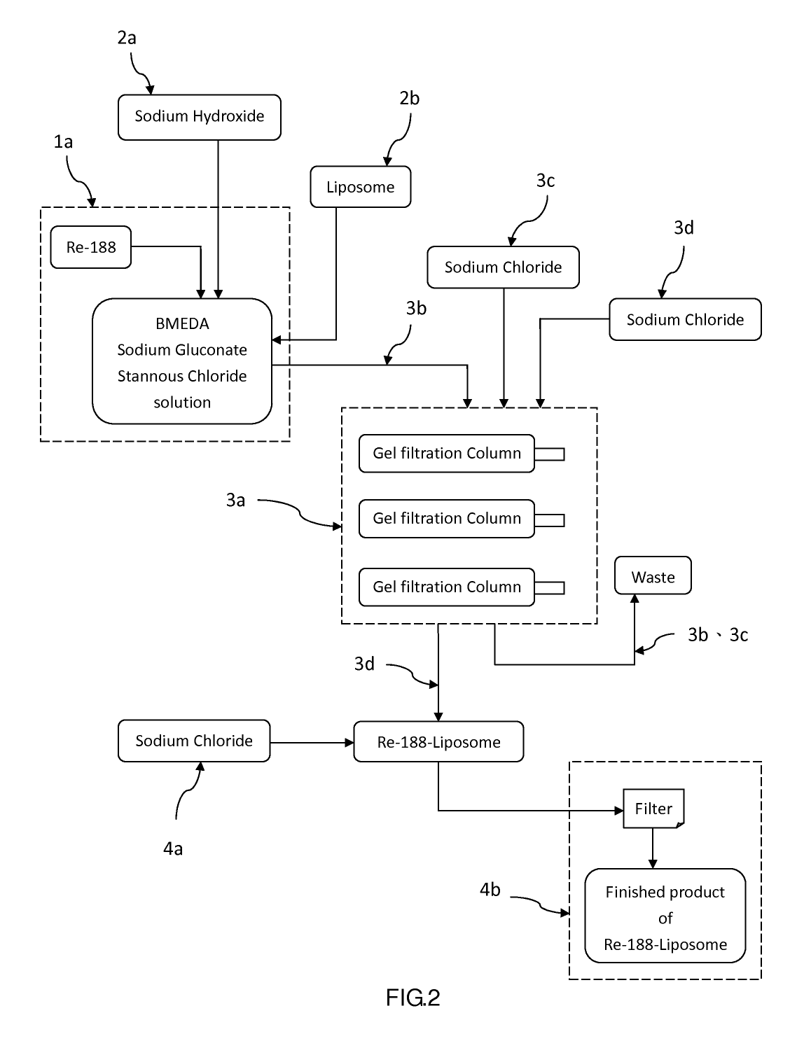

In FIG. 2, a preferred embodiment of automated synthesis method to produce rhenium-188-BMEDA solution is shown, at least comprising steps:

Stage 1: Labeling reaction

Step 1a: charging 77 μL of 0.68M sodium gluconate containing 10% glacial acetic acid solution, 3.08 mg of N,N-bis(2-mercapto ethyl)—N′,N′-diethylethylenediamine, referred to as BMEDA, and 74 μL solution of stannous chloride (SnCl2) with 10 mg/mL modulation ratio into reaction vial G1, and adding nitrogen or helium gas into solution flow paths of micro pumps for pressure regulation, then adding Re-188 solution (radioactivity of approximately 65 mCi˜470 mCi) from the first reagent vial R1 to the reaction vial G1, the reaction taking place at temperature about 80° C. with a stirrer rotating at speed 100-500 rpm to complete the reaction in the reaction vial G1, and the labeling reaction is accomplished with the solution of rhenium-188-BMEDA obtained.

Stage 2: Embedding Reaction

Step 2a: adding 55 μL of equivalent concentration 2N sodium hydroxide from the second reagent vial R2 to reaction vial G1, mixing with rhenium-188-BMEDA solution obtained in step 1a, and stirring the vial G1 with a stirrer rotating at speed 100˜500 rpm to assure uniformity of mixing.

Step 2b: adding 5 mL of liposome from third reagent bottle R3 to the reaction vial in G1, heating the reaction vial G1 to about 60° C. and stirring the reaction vial G1 with a stirrer rotating at speed 100˜500 rpm for 30 minutes to assure uniformity of mixing.

Step 2c: After completion of the reaction, placing the reaction vial G1 at room temperature environment for cooling about 10 minutes, and about 6 ml of 188Re-Liposome solution can be obtained.

Stage 3: Separation and Purification

Step 3a: first using 20 mL of 0.9% sodium chloride injection for washing first to third PD-10 gel filtration column C1, C2, C3, respectively.

Step 3b: adding an equal amount of 2 mL of rhenium-188-liposome solution from the reaction vial G1 to each of PD-10 gel filtration column C1, C2, C3, respectively, and after about 6 mL of the Re-188-liposome solution is completely discharged out of each of the PD-10 gel filtration columns, then adding an equal volume of 1 mL of 0.9% sodium chloride injection to each of PD-10 gel filtration column C1, C2, C3 from the fourth reagent vial R4, respectively, then subject to an amount of 3 mL of the sodium chloride solution being fully discharged as waste solution, conveying the waste solution into waste vial W1 through sixth solenoid valve V6.

Step 3c: after the waste solution being completely discharged, adding an equal volume of 2.5 mL of 0.9% sodium chloride injection to each of PD-10 gel filtration column C1, C2, C3 from the fifth reagent vial R5, respectively.

Step 3d: conveying the discharged about 7.5 mL sodium chloride injection through the sixth solenoid valve V6 into temporary storage vial T1 to complete the process of separation and purification.

Stage 4: Medication and Filtration

Step 4a: adding 0.9% sodium chloride injection from sixth reagent vial R6 to a temporary storage vial T1 to mix with the solution obtained in Step 3d.

Step 4b: filtering the solution obtained from step 4a through micro pump P8 and a sterile membrane F1, and then filling in a sterile glass vial RL1 as a finished product of rhenium-188-Liposome.

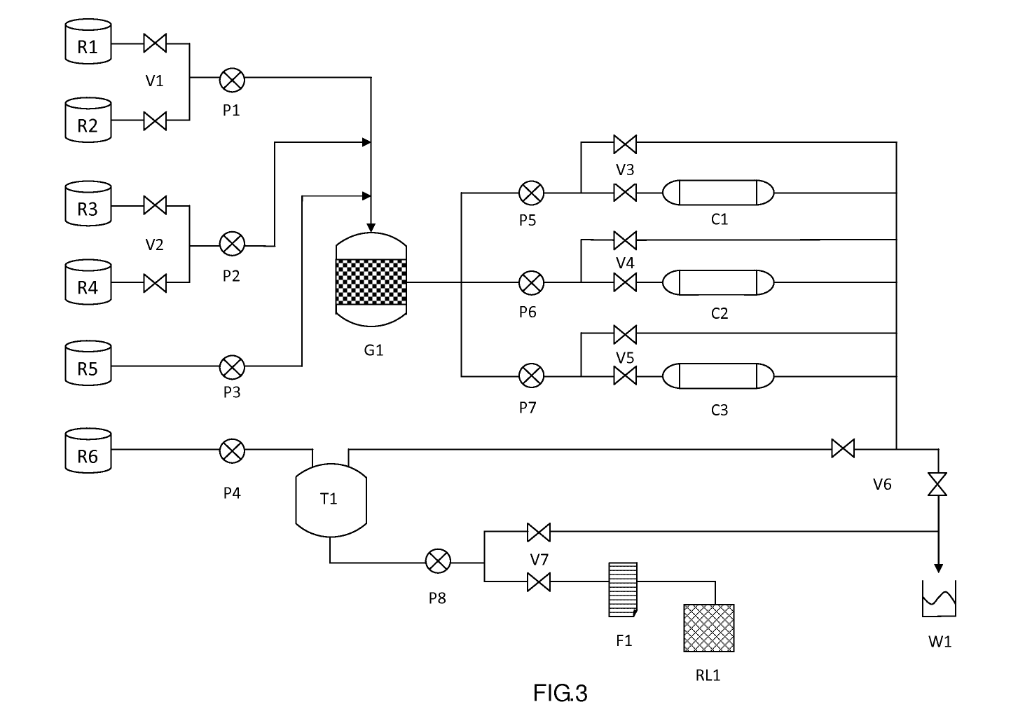

In FIG. 3, a schematic diagram showing a preferred embodiment of synthesis device to produce Re-188-BMEDA solution of the present invention, at least comprising constituent elements: the first to sixth reagent vials R1-R6, the first to seventh three-way solenoid valve V1-V7, the first to third PD-10 gel filtration column C1-C3, the first to eighth micro pump P1-P8, a reaction vial G1, a product vial RL1, a temporary storage vial T1, a filter membrane F1, and a waste vial W1, wherein each element are connected to electric circuit through micro-channel, and controlled with programmable chip to let fluid flow from the reagent vials into the reaction vials in a sequence for reaction through solenoid valves.

The first reagent vial R1 and second reagent vial R2 are being connected to the first solenoid valve V1, and then connected to the reaction vial G1 through the first micro pump P1.

The third reagent vial R3 and fourth reagent vial R4 are being connected to the second solenoid valve V2, respectively, and then connected to the reaction vial G1 through the second micro pump P2. The fifth reagent vial R5 is being connected to the reaction vial G1 through the third micro pump P3. The sixth reagent vial R6 is being connected to the temporary storage vial T1 through the fourth micro pump P4.

The reaction vial G1 is being connected to the micro-pump P5-P7 and the micro-pump P5-P7 are connected to each of PD-10 gel filtration column C1-C3 through the solenoid valve V3-V5, respectively; and each of PD-10 gel filtration column C1 to C3 is connected to the temporary storage vial T1 and waste vial W1 through the sixth solenoid valve V6, respectively, and the temporary storage vial T1 is connected to micro pump P8 and to the waste vial W1 and filter membrane F1 through the seventh solenoid valves V7, respectively, and the filter membrane F1 is connected to a sterile glass product vial RL1.

The foregoing invention has been described in detail by way of illustration and example for purposes of clarity and understanding. It will be apparent to those of ordinary skill in the art that variations, changes, modifications and alterations may be applied to the compositions and/or methods described herein without departing from the true spirit, concept and scope of the invention.

What is claimed is:

1. An automated synthesis device to produce Re-188-BMEDA solution, at least comprising constituent elements: a plurality of reagent vials, three-way solenoid valves, gel filtration columns, micro pumps, a reaction vial, a sterile glass product vial, a temporary storage vial, a filter membrane, and a waste vial, wherein the plurality of vials include first reagent vial R1 and second reagent vial R2 being connected to the reaction vial G1 through first micro pump P1, third reagent vial R3 and fourth reagent vial R4 being connected to the reaction vial G1 through second micro pump P2, fifth reagent vial R5 being connected to the reaction vial G1 through third micro pump P3; and sixth reagent vial R6 being connected to the temporary storage vial T1 through fourth micro pump P4; wherein the reaction vial G1 is connected to gel filtration columns C1-C3 through fifth to seventh micro-pumps P5-P7 and third to fifth solenoid valves V3-V5, respectively; wherein each of the gel filtration columns C1-C3 is connected to the temporary storage vial T1 and waste vial W1 through sixth solenoid valve V6, and wherein the temporary storage vial T1 is connected to eighth micro pump P8 and then to the waste vial W1 and filter membrane F1 through seventh solenoid valve V7, respectively, and wherein the filter membrane F1 is connected to a sterile glass product vial RL1.

2. The automated synthesis device to produce Re-188-BMEDA solution, wherein each element is being connected to electric circuit through micro-channel, and controlled with program built-in chip through one-touch button to let fluid flow from the reagent vials into reaction vials for reaction in a sequence through solenoid valves.

3. The automated synthesis device to produce Re-188-BMEDA solution of claim 1, wherein the first to seventh solenoid valves are three-way solenoid valve.

4. The automated synthesis device to produce Re-188-BMEDA solution of claim 1, wherein the gel filtration columns is PD-10 column.

5. A method of using the automated synthesis device to produce Re-188-BMEDA solution of claim 1, comprising steps:
Step 1a: Charging 77μL of 0.68M sodium gluconate containing 10% glacial acetic acid solution, about 3.08 mg of N,N-bis(2-mercapto ethyl)—N′,N′-diethylethylenediamine, referred to as BMEDA, and 74 μL of solution of stannous chloride (SnCl2) with 10 mg/mL modulation ratio into reaction vial G1, and followed by adding nitrogen or helium gas into solution flow paths of micro pumps for pressure regulation, adding 0.9 mL of Re-188 solution with radioactivity of approximately 65 mCi˜470 mCi to the reaction vial G1 from first reagent vial R1 through micro pump P1, and a reaction taking place at temperature about 80° C. with a stirrer rotating at speed 100˜500 rpm for 60 minutes to complete the reaction in the reaction vial G1, and a labeling reaction is accomplished with 1.05 mL of the solution of rhenium-188-BMEDA obtained;
Step 2a: adding sodium hydroxide from second reagent vial R2 to the reaction vial G1 through micro pump P1, mixing with the rhenium-188-BMEDA solution obtained in step 1a, and stirring the vial G1 with a stirrer rotating at speed 100˜500 rpm to assure uniformity of mixing;
Step 2b: adding 5 mL of liposome from third reagent bottle R3 through micro pump P2 to the reaction vial in G1, heating the reaction vial G1 to about 60° C. and stirring the reaction vial G1 with a stirrer rotating at speed 100˜500 rpm for 30 minutes;
Step 2c: After completion of the reaction, placing the reaction vial G1 at room temperature environment for cooling about 10 minutes, and 188Re-Liposome solution is obtained;
Step 3a: first using 20 mL of 0.9% sodium chloride injection through micro pump P5-P7 for washing first to third PD-10 gel filtration columns C1-C3, respectively;
Step 3b: adding an equal amount of rhenium-188-liposome solution through micro pumps P5-P7 from the reaction vial G1 to each of PD-10 gel filtration columns C1-C3, respectively, and after about 6 mL of the Re-188-liposome solution is completely discharged out of each of the PD-10 gel filtration column C1-C3, then adding an equal volume of 1 mL of 0.9% sodium chloride injection from fourth reagent vial R4 through micro pump P5-P7 to each of PD-10 gel filtration column C1-C3, respectively, then subject to an amount of 3 mL of the sodium chloride solution is fully discharged as waste solution, conveying the waste solution into waste vial W1 through sixth solenoid valve V6;
Step 3c: after the waste solution being completely discharged, adding an equal volume of 2.5 mL of 0.9% sodium chloride injection from fifth reagent vial R5 through micro pumps P5-P7 to each of PD-10 gel filtration column C1-C3, respectively;
Step 3d: conveying the discharged about 7.5 mL of sodium chloride injection through the sixth solenoid valve V6 into temporary storage vial T1 to complete the process of separation and purification;
Step 4a: adding 6.5 mL of 0.9% sodium chloride injection from sixth reagent vial R6 through micro pump P4 to a temporary storage vial T1 to mix with the solution obtained in Step 3d;
Step 4b: filtering the solution obtained from step 4a through micro pump P8 and solenoid valve V7 and sterile membrane F1, and then filling in a sterile glass vial RL1 as a finished product of rhenium-188-Liposome.
